(19) United States
(12) Patent Application Publication
(10) Pub. No.: US 2002/0042536 A1
(43) Pub. Date: Apr. 11, 2002

(54) PREPARATION OF AMINO-PROTECTED LYSINE DERIVATIVES

(76) Inventor: PRASAD S. RAJE, GURNEE, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 09/414,118

(22) Filed: Oct. 8, 1999

(51) Int. Cl. C07C 269/00; C07C 229/00; C07C 259/00

(52) U.S. Cl. 560/24; 562/440; 562/442; 562/562

(57) ABSTRACT

The invention relates to novel processes for preparing differentially protected lysine derivatives via a novel p-anisaldehyde Schiff base intermediate and the intermediate prepared therein.

### TECHNICAL FIELD OF THE INVENTION

The invention relates to novel processes for preparing differentially protected lysine derivatives via a novel p-anisaldehyde Schiff base intermediate and the intermediate prepared therein.

### BACKGROUND OF THE INVENTION

Amino-protected lysine derivatives are a commonly used starting material for various synthetic processes and for the preparation of peptides or peptidic compounds. Formation of undesired impurities and the difficulty in removing those impurities prevent an efficient, robust synthesis of the controlled protection of the N^{α} and N^{ε} amino groups of the lysine residue.

One method that has been described for preparing amino-protected lysine derivatives involves the temporary protection of the N^{α} amino group by copper chelation while the N^{ε} amino group is reacted with an amino-protecting group, for example, “N^{α}-Formyl-L-Lysine in Peptide Synthesis,” Journal of the American Chemical Society, 82, 3727-3732 (1960); and “On the Peptides of L-Lysine,” Journal of the American Chemical Society, 83, 719-722 (1961). Generally, large scale preparation of compounds by these methods can be complicated by a number of factors including, for example, the inconvenience and toxicity of a hydrogen sulfide starting material, the toxicity of a thioacetamide starting material, and the difficult separation of a finished lysine product from a copper sulfide by-product; see “A Procedure for the Large Scale Preparation of N^{ε}-Alloc-Lysine and N^{ε}-Alloc-N^{α}-Fmoc-Lysine,” Synthetic Communications 23(1), 49-53 (1993).

In another method, the N^{ε} amino group is temporarily protected as a benzilidine Schiff base while the N^{α} amino group is protected with another suitable amino-protecting group, as detailed in “Some Schiff Bases of Free Amino Acids,” Journal of the American Chemical Society, 69, 1377-1380 (1947); “Studies on Schiff Bases in Connection with the Mechanism of Transamination,” Journal of the American Chemical Society, 76, 5589-5597 (1954); Journal of Organic Chemistry 33(3), 1261-1264 (1968); “Improved Syntheses of N^{ε}-Tert-Butyloxycarbonyl-L-Lysine and N^{α}-Benzyloxycarbonyl-N^{ε}-Tert-Butyloxycarbonyl-L-Lysine,” Synthetic Communications 11(4), 303-314 (1981). The moderate selectivity of the benzilidine Schiff base preparation results in the formation of an impure diprotected lysine material which is difficult to purify due to the inefficient processes available for removing the formed impurities. The use of the impure material on a large-scale peptide synthesis causes the production of undesired side products. Accordingly, there remains a need for an efficient, robust synthesis of differentially protected lysine derivatives.

It would be beneficial to provide a process demonstrating improved selectivity of the desired N^{α} or N^{ε} amino group. An advantageous process would allow for the controlled selection of the desired amino-protecting group at the N^{α} and N^{ε} amino moieties. A favorable synthesis would also allow for eliminating unnecessary impurities in process while providing differentially protected lysine amino acids in robust yield with high purity.

### SUMMARY OF THE INVENTION

It has been surprisingly found that use of a p-anisaldehyde Schiff base allows for the protection of the N^{α} and N^{ε} amino groups of lysine, or a derivative thereof, with a different protecting group at each nitrogen atom. A process which proceeds via a p-anisaldehyde Schiff base intermediate demonstrates improved selectivity for the desired amino group with a minimum formation of impurities. The undesirable impurities that are formed can be efficiently removed in process.

In one aspect, therefore, the present invention relates to a process for preparing a compound of the formula:

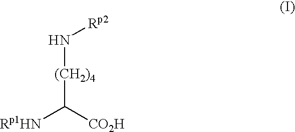

or a salt or ester thereof, wherein R^{p1} and R^{p2} are independently selected from hydrogen or an amino-protecting group, comprising the steps of:

(a) treating lysine, or a salt thereof, with p-anisaldehyde optionally in the presence of a base;

(b) protecting the N^{α} amino moiety with an amino-protecting group;

(c) hydrolyzing the compound obtained in step (b) in the presence of an acid; and

(d) optionally protecting the N^{ε} amino moiety of the compound obtained in step (c) with an amino-protecting group.

In another aspect, the invention relates to a process for preparing a compound of formula (I), as defined above, comprising reacting a compound of the formula:

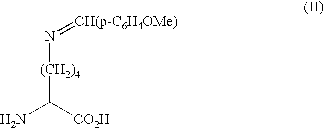

with an amino-protecting group, hydrolyzing the compound obtained therefrom and protecting any unprotected amino group.

In yet another aspect, the invention relates to a compound of the formula:

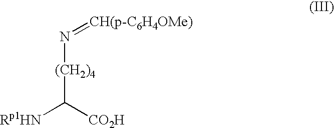

or a salt or ester thereof, wherein R^{p1} is hydrogen or an amino-protecting group.

### DETAILED DESCRIPTION OF THE INVENTION

The present invention relates to a process for preparing mono- or di-protected lysine derivatives. The derivatives can be differentially protected at the N^{α} or the N^{ε} amino groups of the lysine derivative. Synthesis of the differentially protected lysine derivatives is accomplished via the preparation of a p-anisaldehyde lysine Schiff base. The process involving the p-anisaldehyde Schiff base allows for the minimum formation of impurities. The removal of impurities from the reaction mixture can be easily achieved.

The term “amino-protecting group” as used herein refers to a substituent that protects an amino functionality against undesirable reactions during synthetic procedures. Amino-protecting groups are typically acyl, urea, urethane, nitroso, nitro, sulphenyl, sulphonyl, sulfonic acid, or trialkylsilyl. Examples include acetyl, carbobenzyloxy (also benzyloxycarbonyl or carbobenzoxy), formyl, t-butyloxycarbonyl, fluorenylmethyloxycarbonyl, 2-nitrophenylsulfenyl, methanesulfonyl, p-toluenesulfonyl, and the like. A thorough discussion of amino-protecting groups disclosed in Protective Groups in Organic Synthesis, John Wiley & Sons, NY, 1981, by T. W. Greene and P. G. M. Wuts, which is incorporated herein by reference.

The term “amino-protecting reagent” as used herein refers to a compound that reacts with the amino functionality to give a protected amino group, which can be represented by the formula —NHR^{p}, wherein R^{p} represents an amino-protecting group as previously described above. For example, the reagent benzyloxycarbonyl chloride affords the benzyloxycarbonyl protecting group. Other types of amino-protecting reagents include, but are not limited to, acylating reagents, sulfonylating reagents, sulfenylating reagents, urea and urethane-type reagents, nitroso derivatives, nitro derivatives, and trialkylsilyl reagents. It will be obvious to those skilled in the art that individual reagents or reagent combinations may be preferred for specific compounds and reaction conditions, depending upon such factors as the solubility of reagents, reactivity of reagents, preferred temperature ranges and suitable conditions for removing the protecting group or excess protecting reagent. Various amino-protecting reagents have been described by Greene & Wuts in Protective Groups in Organic Synthesis.

The term “aprotic solvent” as used herein refers to a solvent that is relatively inert to proton activity, for example, not acting as a proton-donor. Examples include hydrocarbons, such as hexane and toluene, for example halogenated hydrocarbons, such as for example, methylene chloride, ethylene chloride, chloroform, and the like, heteroaryl compounds, such as, for example, tetrahydrofuran and N-methylpyrrolidinone, and ethers, such as diethyl ether and bis-methoxymethyl ether. Such compounds are well known to those skilled in the art, and the individual solvents or mixtures thereof may be preferred for specific compounds and reaction conditions, depending upon such factors, for example, as the solubility of reagents, reactivity of reagents and preferred temperature ranges. Further discussions of aprotic solvents may be found in organic chemistry textbooks or in specialized monographs, such as: Organic Solvents Physical Properties and Methods of Purification, 4th ed., edited by John A. Riddick et al., Vol. II, in the Techniques of Chemistry Series, John Wiley & Sons, NY, 1986.

The term “polar aprotic solvent” as used herein refers to an aprotic solvent, as described above, having a relatively high dielectric constant. Polar aprotic solvents generally lack hydroxyl groups or a similar hydrogen-bonding functionality. Examples of polar aprotic solvents include, acetone, acetonitrile, dimethyl sulfoxide, N,N-dimethylformamide, N-methylpyrrolidone, hexamethylphosphoric triamide, tetrahydrofuran, dimethoxyethane, and the like. Further discussions of the characteristics of polar aprotic solvents can be found in Advanced Organic Chemistry, 3rd. edition, Plenum Press, NY, 1990 by Francis A. Carey and Richard J. Sundberg.

The term “differentially protecting” or “differentially protected” as used herein refers to the placement of dissimilar protecting groups on the free, unprotected amino groups of a compound having at least two, and preferably two, amino groups. In the case wherein the diaminated compound is lysine, the N^{α} and the N^{ε} amino groups are reacted with various amino-protecting reagents as described above in a suitable manner to afford distinct protecting groups on the N^{α} and N^{ε} nitrogen atom.

The term “salt or ester thereof” as used herein refers to the acid addition salt of a compound of the invention and a compound derived from the condensation of a compound of the invention with an acid or an alcohol, respectively.

The term “salt” refers to the inorganic and organic acid addition salts of the compound of the present invention. The preparation of a salt is well known in the art and can be accomplished, for example, in situ during the final isolation and purification of the compounds of the invention; by reacting the free base function with a suitable organic acid; or by using other methods used in the art, such as ion exchange. Examples of some non-toxic acid addition salts are the salts formed from a reaction with inorganic acids, such as hydrochloric acid, hydrobromic acid, phosphoric acid, sulfuric acid and perchloric acid, or with organic acids, such as acetic acid, oxalic acid, maleic acid, tartaric acid, citric acid, succinic acid or malonic acid. Other salts include adipate, alginate, ascorbate, aspartate, benzenesulfonate, benzoate, bisulfate, borate, butyrate, camphorate, camphorsulfonate, citrate, cyclopentanepropionate, dicyclohexylamine, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. A detailed discussion of salts can be found in J. Pharmaceutical Sciences, 66: 1-19 (1977) by S. M. Berge, et al., which is incorporated herein by reference.

The term “ester” refers to a compound derived from the condensation of a compound of the invention with an acid or an alcohol. Examples of esters of the compounds of this invention include inorganic or organic esters derived from condensation with an inorganic or organic acid, respectively. An ester, for example, C₁ to C₆ alkanoyl esters wherein the alkanoyl group is a straight or branched chain. Hydroxysuccinimide esters, and the like, may be prepared according to conventional methods using a compound of the invention.

Certain abbreviations have been used in the throughout the description and in the schemes and examples for the ease of describing the invention. As used herein, the following abbreviations denote the following: Boc for tert-butyloxycarbonyl; DCC for dicyclohexyl-carbodiimide; DCHA for dicyclohexylamine; DCU for N,N-dicyclohexylurea; EtOAc for ethyl acetate; EtOH for ethanol; HCl for hydrochloric acid; IPA for isopropyl alcohol; KHSO₄ for potassium hydrogen sulfate; LiOH for lithium hydroxide; NaOH for sodium hydroxide; NHS for N-hydroxysuccinimide; OSu for N-hydroxysuccinimide ester; THF for tetrahydrofuran; Z (or Cbz) for benzyloxycarbonyl; and Z-Cl for benzyloxycarbonyl chloride.

An example of the inventive process is presented below in accordance with the following Scheme 1:

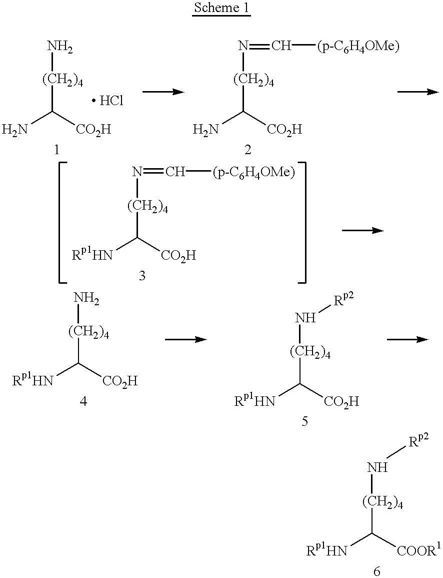

In accordance with Scheme 1, a lysine derivative 1 is treated with p-anisaldehyde to form a Schiff base 2. The reaction can be accomplished with a lysine free amino acid or a derivative thereof optionally in the presence of base. Exemplary derivatives of the lysine amino acid are an acid addition salt of lysine or lysine hydrate. Preferably, the reaction is accomplished with the acid addition salt. Suitable salts for the reaction include, lysine monohydrochloride, lysine dihydrochloride, and the like. Lysine monohydrochloride is the preferred salt for the reaction.

A commercially available p-anisaldehyde reagent (Aldrich, Milwaukee, Wis.) can be added to lysine or a lysine derivative. A total amount of about 0.9 to about 1.2 molar equivalents of p-anisaldehyde can be added to reaction mixture for each mole of lysine. Preferably, a total amount of about 1.05 equivalents of p-anisaldehyde reagent are used for each mole of the lysine or lysine derivative starting material. The p-anisaldehyde reagent is preferably added to the reaction mixture in portions.

Where a lysine derivative is used, the reaction can be accomplished in the presence of an organic or inorganic base to generate the free amine of the lysine derivative. Although carbonates and organic amine bases may be suitable for the reaction, it is preferred that the base is a metal hydroxide base. Exemplary metal hydroxide bases for the reaction include, but are not limited to, lithium hydroxide, sodium hydroxide, magnesium hydroxide, cesium hydroxide, and the like. Preferably, about 0.95 to about 1.15 molar equivalent of base is reacted with each mole of the lysine or lysine derivative starting material. The preferred base for the reaction is lithium hydroxide.

The reaction proceeds more efficiently when accomplished at temperatures from about −5° C. to about room temperature. Preferably, the reaction is accomplished at about 0° C.

Formation of the p-anisaldehyde Schiff base 2 allows for the protection of the N^{α} amino group of the Schiff base 3, wherein R^{p1} is hydrogen or an amino-protecting group. The Schiff base 2 can be treated with a suitable amino-protecting reagent in the presence of base.

The amino-protecting reagents suitable for the reaction typically comprise a reagent suitable for preventing the reaction of the nitrogen atom of the N^{α} or the N^{ε} unprotected amine. Suitable protecting groups for the reaction include, but are not limited to, acyl, urea, urethane, nitroso, nitro, sulphenyl, sulphonyl, sulfonic acid, trialkylsilyl, and the like. Preferred amine-protecting groups suitable for the reaction are formyl, acetyl, benzyloxycarbonyl, t-butyloxycarbonyl, fluorenylmethyloxycarbonyl, methanesulfonyl, p-toluenesulfonyl, 2-nitrophenylsulfenyl, and the like. Exemplary types of reagents for placing the amino-protecting groups on the unprotected amine include, but are not limited to, acylating reagents, sulfonylating reagents, sulfenylating reagents, urea and urethane-type reagents, nitroso derivatives, nitro derivatives, trialkylsilyl reagents, and the like. Preferred amino-protecting reagents are selected from di-tert-butyl dicarbonate, t-butyl chloroformate (not commercially available), 2-(t-butoxycarbonyloxyimino)-2-phenylacetonitrile, N-t-butoxy-carbonyloxysuccinimide, 1-(t-butoxycarbonyl)imidazole, and benzyloxycarbonyl chloride. Additional amino-protecting groups for the reaction are described in Protective Groups in Organic Synthesis, John Wiley & Sons, NY, 1981, by Theodora W. Greene and Peter G. M. Wuts.

The amount of the amine-protecting reagent can vary depending on which amine-protecting reagent is used. Typically, the reaction can be accomplished with from about 1.0 to about 4.0 molar equivalents of the amino-protecting reagent relative to one molar equivalent of the Schiff base. Preferably, about 1.0 to about 1.5 molar equivalents of the amino-protecting reagent are used. It is preferred that an inorganic or organic base is added in portions while maintaining the reaction at a suitable temperature. Typically, the reaction proceeds in a more efficient manner when the temperature of the reaction mixture is maintained near or below −5° C.

The reaction can be accomplished in the presence of an organic or inorganic base. Preferably, the inorganic base is a metal hydroxide base, such as lithium hydroxide, sodium hydroxide, magnesium hydroxide, cesium hydroxide, and the like, or a mixture thereof. Amines can be the suitable organic base. Carbonates may also be suitable for the reaction.

Suitable solvents are alcoholic solvents, such as methanol, ethanol, isopropanol, and the like, or a mixture thereof. Other solvents suitable for the reaction include, but are not limited to, tetrahydrofuran, isopropyl acetate, methyl t-butyl ether, ethyl ether, and the like, or a mixture thereof.

Direct hydrolysis without isolating the intermediate 3 affords the N^{α}-amino-protected lysine derivative 4 with other impurities under acidic conditions. Suitable acids are organic or inorganic acids. Exemplary organic acids include, but are not limited to, acetic acid, benzoic acid, citric acid, 3-nitrobenzoic acid, 4-nitrobenzoic acid, 4-aminobenzoic acid, 2-methylbenzoic acid, propanoic acid, butanoic acid, and the like. Inorganic acids suitable for the reaction include, but are not limited to, hydrochloric acid, sulfuric acid, phosphoric acid, nitric acid, p-toluenesulfonic acid, and the like.

Impurities which have been identified in the reaction mixture include the compounds having the formula:

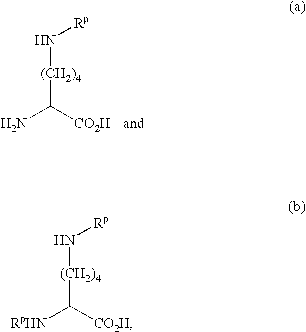

wherein R^{p} is an amino-protecting group. To obtain a differentially protected N^{α}-amino-N^{ε}-amino-protected lysine derivative, it is preferred that the N^{ε}-amino-protected lysine derivative (a) and the diprotected N^{α},N^{ε}-amino-protected lysine derivative (b) are removed from the reaction mixture.

The N^{ε}-amino-protected lysine derivative can be selectively removed by adjusting the pH of the reaction mixture to a pH between about 2.0 and about 3.5, and more preferably between about 3.0 and about 3.5. It is preferred that the reaction mixture is maintained below room temperature, preferably between about −5° C. and 0° C. during adjustment of the pH. The solid formed in the resulting reaction mixture has been characterized as a precipitate of the impurities (a). The precipitate can be easily removed from the reaction mixture by filtration.

The diprotected N^{α},N^{ε}-amino-protected lysine derivative (b) can be easily removed from the reaction mixture by washing with a water immiscible solvent. Exemplary solvents are dichloromethane, ethyl acetate, diethyl ether, methyl t-butyl ether, and the like.

The lysine derivative 4 can be reacted with an amino-protecting reagent in the presence of a base to provide 5, wherein R^{p2} is hydrogen or an amino-protecting group. The amino-protecting reagent can be selected from the group as previously described. It is preferred that the amino-protecting reagent selected provides an amino-protecting group that is different from the amino-protecting reagent for the preparation of 3. Where the amino-protecting groups for protecting the N^{α} and the N^{ε} amino groups are different, the lysine derivative can be referred to as a differentially protected lysine derivative as previously described.

The base for the reaction can be selected from the group of bases as previously described for the preparation of 3. The preferred base is sodium hydroxide. The reaction can be carried out in an aprotic solvent, preferably tetrahydrofuran. In a most preferred reaction, a di-t-butyldicarbonate protecting reagent is reacted with lysine derivative 4 in tetrahydrofuran in the presence of sodium hydroxide. Preferably, the reaction is accomplished at low temperatures from about 0° C. to about 5° C.

It is preferred that the protection of 4 is accomplished with less than one molar equivalent of the amine-protecting reagent relative to one mole of the protected Schiff base. It is more preferred that the reaction is accomplished with from about 0.85 to about 0.95 molar equivalents of the amine-protecting reagent.

The process of the invention provides an efficient synthesis for preparing lysine derivatives wherein the N^{α} and the N^{ε} amino groups are protected with distinct amino-protecting groups. The high selectivity of steps during the process allows for robust, large scale synthesis of any mono- or di-protected lysine derivative.

For convenience during characterization, the protected lysine derivatives can be prepared as a dicyclohexylamine (DCHA) salt, if desired. Briefly, a solution of a mono- or di-protected lysine derivative is treated with dicyclohexylamine in an inert atmosphere, cooled and filtered. Methods for preparing DCHA salts of lysine derivatives are known in the art and have been described in Synthetic Communications, 11(4), 303-314 (1981).

The lysine derivative 5 can be coupled with a suitable ester or organic group in accordance with methods readily available in the art. Typically, the reaction is carried out with a suitable coupling reagent. Preferably, the coupling agent is commonly used for preparing an amide bond. A preferred coupling reagent is dicyclohexylcarbodiimide (DCC). In a preferred reaction, DCC is coupled with N-hydroxysuccinimide (NHS) in an aprotic solvent. Preferably, one molar equivalent of DCC is used for one mole of starting material 5. Preferably, one molar equivalent of NHS is used for one mole of starting material 5. A further discussion of coupling reactions of amino acids has been described by M. Bodanszky and B. Trost, Ed., Principles of Peptide Synthesis, 2nd ed., Springer-Verlag Inc., NY, 1993, which is herein incorporated by reference.

In another aspect, the invention relates to a process for preparing a compound of formula (I), as defined above, comprising reacting a compound of the formula:

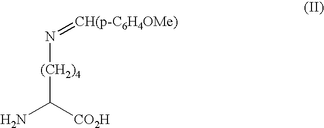

with an amino-protecting group, hydrolyzing the compound obtained therefrom and protecting any unprotected amino group.

In yet another aspect, the invention relates to a compound of the formula:

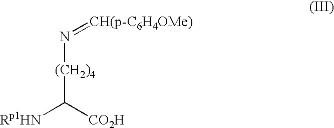

or a salt or ester thereof, wherein R^{p1} is hydrogen or an amino-protecting group.

Compounds and processes of the invention provide useful starting materials for the synthesis of peptide and peptidic compounds. In particular, the compounds are useful for the synthesis of a variety of pharmaceutical compounds including, but not limited to, antibiotics, anticancer agents, antifungal agents, and antithrombolytics. However, the compounds of the invention, including salts and esters obtained therefrom, can be successfully incorporated into the synthesis of various non-pharmaceutical peptide or peptidic compounds.

The compounds and processes of the present invention will be better understood in connection with the following examples, which are intended as an illustration of and not a limitation upon the scope of the invention. All reagents are commercially available and can be obtained from Aldrich Chemical Company (Milwaukee, Wis., U.S.A.), unless otherwise noted.

### EXPERIMENTAL

Step 1(a): N^{ε}-anisilidine-lysine [Lys[N═CH-(p-C₆H₄OMe)]-OH, (2)]

To a 2.0 LiOH (2N) solution at room temperature was added lysine monohydrochloride (696 g) (1) and reaction mixture was cooled to below 3° C. p-Anisaldehyde (545 g) was added in 10 portions of about 50 grams each. The reaction mixture was stirred for 3 hours maintaining temperature below 5° C. The reaction mixture (thick paste) was allowed to stand at below 5° C. overnight. Then the paste was filtered with aid of cold 1.0 L acetonitrile and the wet cake was used in the next step without further purification.

mp 179-180° C.; νₘₐₓ (neat): 2918, 2820, 2813, 1647, 1602, 1576, 1508, 1439, 1403, 1351, 1307, 1299, 1251, 1158, 1105, 1028, 830, 665 cm⁻¹; ¹H NMR (500 MHz, D₂O+drop of HOAc) δ1.32-1.48 (2H, m), 1.62-1.68 (2H, m), 1.78-1.88 (2H, m), 2.95 (2H, t, J 7.6 Hz), 3.68 (1H, t, J 6.1 Hz), 3.85 (3H, s), 7.05-7.08 (2H, m), 7.84-7.86 (2H, m), 9.87 (1H, s).

Step 1(b): N^{α}-benzyloxycarbonyl-lysine [Z-Lys-OH, (4)]:

Wet cake of Schiff base (2) was suspended in 500 ml EtOH and cooled to −15° C. A solution of 3.0 L NaOH (1N) and 1.5 L EtOH previously cooled to −15° C. was added with vigorous stirring to the suspension of Schiff base. The benzyloxycarbonyl (Z-Cl) and a cold solution of a mixture of EtOH and NaOH (−15° C., 2.3 L EtOH and 4.6 L NaOH (1N)) was added in portions. The Z-Cl and the EtOH/NaOH solution were added at a rate of addition such that the internal temperature of the reaction mixture was maintained at less than about −5° C. After complete addition, the reaction mixture was stirred for about 30 minutes allowed to warm to a temperature of about −4° C. to provide the intermediate (3). The pH of the reaction mixture was then adjusted to ˜2.0 by adding concentrated HCl, which hydrolyzes the intermediate. Ethanol in the reaction mixture was then distilled at around 50° C. The aqueous layer was then extracted with EtOAc (4×250 ml) followed by concentrating the aqueous layer to ˜1.6 L. The pH of the aqueous layer was then adjusted to between 3.0-3.5, cooled to 2-3° C. and stirred for 10 hours. Any precipitates formed were filtered and the aqueous layer used in the next step without further purification.

Step 1(c): N^{α}-benzyloxycarbonyl-N^{ε}-tert-butyloxycarbonyl-lysine [Z-Lys(Boc)-OH, (5)]:

To a solution of Z-Lys-OH (4) (2.1 L aqueous solution, 683 gm estimated amount from 80% yield), THF (1.8 L) was added with stirring and the mixture cooled down to 0-5° C. in an ice bath. It was treated with 1N NaOH solution (2.437 L) and the temperature of the reaction mixture readjusted to 0-5° C. A solution of di-tert-butyl dicarbonate (Boc-anhydride, 532 gm in 200 mL dry THF) was then added portion wise with vigorous mixing and maintaining temperature below 5° C. The pH was also maintained at approximately pH 10 by adding 1N NaOH. The reaction mixture was stirred at the same temperature for one hour then allowed to come to room temperature and stirred overnight. The pH was kept basic (pH ˜9-10). The reaction mixture was concentrated in vacuo to remove all the THF. The resulting aqueous solution was covered with ethyl acetate (2 L) and the pH of the mixture was adjusted to pH 2-3 using 20% KHSO₄ with stirring and cooling in an ice bath. The reaction mixture was mixed well and the ethyl acetate layer was separated out. The aqueous layer was extracted again with more ethyl acetate (2×1.5 L). Ethyl acetate extracts were pooled and washed with water (2 L, 1.5 L and 1.2 L portions) until a neutral pH (approximately pH 7) was obtained. Ethyl acetate was concentrated in vacuo to give a thick oil. The residue was dried further in vacuo for four hours to yield 727.26 gm product. HPLC 91% pa.

Step 1(d): N^{α}-benzyloxycarbonyl-N^{ε}-tert-butyloxycarbonyl-lysine N-hydroxysuccinimide ester [Z-Lys(Boc)-OSu, (6)]

The oily product obtained above (5) (727 gm) and N-hydroxysuccinimide (242 gm) were taken in dry THF (1.5 L). It was mixed well to dissolve all solid and cooled below 5° C. Dicyclohexylcarbodiimide solution (DCC, 435 gm in 500 mL dry THF) was added dropwise to the mixture with stirring and maintaining temperature below 5° C. More dry THF (500 mL) was used to transfer all the DCC. The reaction mixture was stirred for one hour at 0-5° C. and then allowed to stand in the refrigerator overnight. The DCC was separated by filtration and the cake was washed with more THF (700 mL). The filtrate was concentrated in vacuo to an oil. The oil was mixed with heptane (500 mL) and concentrated to dryness (2X). The oil was then dissolved in isopropyl alcohol (IPA, 1.5 L) by heating (50° C.). Any solid separated was filtered and the filtrate seeded with some pure Z-Lys(Boc)-OSu. The reaction mixture was stirred well and cooled in an ice bath. After approximately 30 minutes, the solids started separating from the solution. The chilled reaction mixture was stirred for one hour and then left in the refrigerator overnight. The solids became quite hard. One liter of cold IPA was used to break the solids. The slurry so obtained was stirred in an ice bath for two hours. The product was filtered and the cake washed with the cold IPA (500 mL). The cake was dried in vacuo (35° C.). The weight of the product obtained was 662 gm. HPLC 93.6%. A portion of above Z-Lys(Boc)-OSu (100 gm) was recrystallized from IPA (800 mL) to give 83 gm pure Z-Lys(Boc)-OSu. HPLC purity 99% pa. [α]²⁵D -17° (c 2.00, Dioxane).

What is claimed is:

1. A process for preparing a compound of the formula: 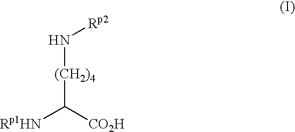
or a salt or ester thereof, wherein R^{p1} and R^{p2} are independently selected from hydrogen or an amino-protecting group, comprising the steps of:
(a) treating lysine, or a salt thereof, with p-anisaldehyde optionally in the presence of a base;
(b) protecting the N^{α} amino moiety with an amino-protecting group;
(c) hydrolyzing the compound obtained in step (b) in the presence of an acid; and
(d) optionally protecting the N^{ε} amino moiety of the compound obtained in step (c) with an amino-protecting group.

2. The process according to claim 1, comprising preparing a N-hydroxysuccinimide ester from a compound of formula (I).

3. The process according to claim 1, comprising preparing a dicyclohexylamine salt of a compound of formula (I).

4. The process for preparing a compound according to claim 1, wherein the amino-protecting reagent is selected from acylating reagents, sulfonylating reagents, sulfenylating reagents, urea and urethane-type reagents, nitroso derivatives, nitro derivatives, and trialkylsilyl reagents.

5. The process according to claim 4, wherein the amino-protecting reagent is di-tert-butyl dicarbonate, t-butyl chloroformate, 2-(t-butoxycarbonyloxyimino)-2-phenyl-acetonitrile, N-t-butoxy-carbonyloxysuccinimide, 1-(t-butoxycarbonyl)imidazole, or benzyloxycarbonyl chloride.

6. The process according to claim 5, wherein the amino-protecting reagent is di-tert-butyl dicarbonate or benzyloxycarbonyl chloride.

7. The process according to claim 1, wherein R^{p1} and R^{p2} are independently selected from acyl, urea, urethane, nitroso, nitro, sulphenyl, sulphonyl, sulfonic acid, or trialkylsilyl.

8. The process according to claim 7, wherein R^{p1} and R^{p2} are independently selected from formyl, acetyl, benzyloxycarbonyl, t-butyloxycarbonyl, fluorenylmethyloxycarbonyl, methanesulfonyl, p-toluenesulfonyl, and 2-nitrophenylsulfenyl.

9. The process according to claim 1, wherein the base in step (a) is a carbonate, an amine, or a metal hydroxide.

10. The process according to claim 9, wherein the base is lithium hydroxide, sodium hydroxide, magnesium hydroxide, or cesium hydroxide.

11. The process according to claim 1, wherein step (b) comprises reacting the p-anisaldehyde Schiff base with an amino-protecting reagent.

12. The process according to claim 11, further comprising adjusting the pH of the reaction mixture to a pH between about 2.0 and about 3.5.

13. The process according to claim 12, wherein the pH is between 3.0 and 3.5.

14. The process according to claim 13, wherein the temperature of the reaction mixture is maintained at a temperature of about −5° C. to about 0° C. during the adjustment of the pH.

15. The process according to claim 1, wherein the acid in step (c) is an organic or inorganic acid.

16. The process according to claim 15, wherein the organic acid is acetic acid, benzoic acid, citric acid, 3-nitrobenzoic acid, 4-nitrobenzoic acid, 4-aminobenzoic acid, 2-methylbenzoic acid, propanoic acid, or butanoic acid.

17. The process according to claim 15, wherein the inorganic acid is hydrochloric acid, sulfuric acid, phosphoric acid, nitric acid, or p-toluenesulfonic acid.

18. The process according to claim 1, wherein the amount of the amino-protecting reagent in step (d) is less than one molar equivalent of the compound obtained in step (c).

19. The process according to claim 18, wherein the amount of the amino-protecting reagent is from about 0.85 to about 0.95 molar equivalent relative to the compound obtained in step (c).

20. A process for preparing a compound of the formula: 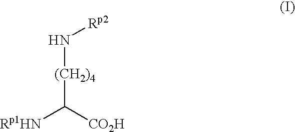
wherein R^{p1} and R^{p2} are hydrogen or an amino-protecting group, comprising reacting a compound of the formula: 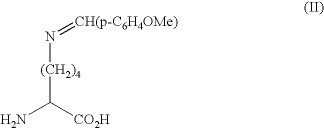
with an amino-protecting group, hydrolyzing the compound obtained therefrom and protecting any unprotected amino group.

21. A compound of the formula: 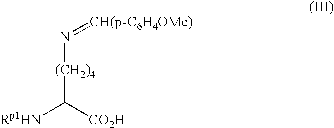
or a salt or ester thereof, wherein R^{p1} is hydrogen or an amino-protecting group.
